(19) United States
(12) Patent Application Publication
(10) Pub. No.: US 2004/0116930 A1
(43) Pub. Date: Jun. 17, 2004

(54) Bone plates

(76) Inventors: Shawn W. O'Driscoll, Rochester, MN (US); David G. Jensen, Troutdale, OR (US)

Correspondence Address: KOLISCH HARTWELL, P.C., 520 S.W. YAMHILL STREET, SUITE 200, PORTLAND, OR, 97204, US

(21) Appl. No.: 10/731,173

(22) Filed: Dec. 8, 2003

Related U.S. Application Data
(63) Continuation of application No. PCT/US02/18623, filed on Jun. 10, 2002.

(51) Int. Cl. A61B 17/56

(52) U.S. Cl. 606/69

(57) ABSTRACT

Sets of bone plates and components thereof for use with particular bones or regions of bones, such as periarticular regions of an elbow. These bone plates may be configured for easy identification, easy fitting, and/or as anatomical templates, among others.

### CROSS-REFERENCES TO PRIORITY APPLICATIONS

This application is a continuation of PCT Patent Application Serial No. PCT/US02/18623, filed Jun. 10, 2002, which, in turn, is based upon and claims the benefit under 35 U.S.C. § ¹¹⁹(e) of U.S. Provisional Patent Application Serial No. 60/297,008, filed Jun. 8, 2001. Each of these priority patent applications is incorporated herein by reference in its entirety for all purposes.

### FIELD OF THE INVENTION

The invention relates to bone plates. More particularly, the invention relates to sets of bone plates and components thereof for use with particular bones or regions of bones, such as an elbow region.

### BACKGROUND OF THE INVENTION

The human skeleton is composed of 206 individual bones that perform a variety of important functions, including support, movement, protection, storage of minerals, and formation of blood cells. These bones can be grouped into two categories, the axial skeleton and the appendicular skeleton. The appendicular skeleton includes among others the long bones Of the upper and lower limbs, including the humerus, radius, and ulna.

To ensure that the skeleton retains its ability to perform its important functions, and to reduce pain and disfigurement, fractured bones should be repaired promptly and properly. Typically, fractured bones are treated using fixation devices, which reinforce the fractured bone and keep it aligned during healing. Fixation devices may take a variety of forms, including casts for external fixation and bone plates for internal fixation, among others.

Bone plates are sturdy, typically metal, plates that may be custom contoured (i.e., bent) by a surgeon to conform to a region of bone spanning a fracture and then fastened to the bone on both sides of the fracture using a suitable fastener, such as one or more screws and/or wires, to hold the fractured bone together during and/or after healing. Bone plates may be provided in various lengths, widths, and shapes to accommodate various sizes and shapes of bones.

Bone plates are considered the treatment of choice for many fractured bones, permitting an early return to motion. However, bone plates suffer from a number of shortcomings. In particular, setting a fracture in some bones, such as the distal end of the humerus, may require the use of more than one bone plate, particularly if the fracture and/or the affected region of bone is complex. Moreover, setting a fracture using more than one plate may be complicated if the different plates are difficult to distinguish and/or need to be contoured to a complex shape prior to use, especially if time is of the essence, as in an operating room.

### SUMMARY OF THE INVENTION

The invention provides sets of bone plates and components thereof for use with particular bones or regions of bones, such as periarticular regions of an elbow. These bone plates may be configured for easy identification, easy fitting, and/or as anatomical templates, among others.

### BRIEF DESCRIPTION OF THE DRAWINGS

FIG. 1 is a lateral view of a left elbow region fixated with embodiments of precontoured bone plates for the lateral and medial condyles of the distal humerus, and the olecranon of the proximal ulna, in accordance with aspects of the invention.

FIG. 2 is a medial view of the left elbow region of FIG. 1 fixated as in FIG. 1, but fixated also with an embodiment of a precontoured bone plate for the coronoid of the proximal ulna, in accordance with aspects of the invention.

FIG. 3 is a posterior view of the distal humerus from the elbow region of FIG. 1, fixated as in FIG. 1, but with an embodiment of a posterior bone plate applied in lieu of the plate for the lateral condyle.

FIG. 4 is a top plan view of an embodiment of a lateral condyle bone plate for fixing lateral-distal portions of a left humerus, particularly the lateral condyle, constructed in accordance with aspects of the invention.

FIG. 5 is a side elevation (profile) view of the bone plate of FIG. 4.

FIG. 6 is a bottom view of the bone plate of FIG. 4.

FIG. 7 is a cross-sectional view of the bone plate of FIG. 4, viewed generally along line 7-7 of FIG. 4.

FIG. 8 is another cross-sectional view of the bone plate of FIG. 4, viewed generally along line 8-8 of FIG. 4.

FIG. 9 is a top plan view of a right-handed embodiment of the lateral condyle bone plate of FIG. 4, constructed in accordance with aspects of the invention.

FIG. 10 is a top plan view of another embodiment of a lateral condyle bone plate, with a longer shaft-anchor portion than in the embodiment of FIG. 4, constructed in accordance with aspects of the invention.

FIG. 11 is a side elevation (profile) view of the bone plate of FIG. 10.

FIG. 12 is a bottom view of the bone plate of FIG. 10.

FIG. 13 is a top plan view of yet another embodiment of a lateral condyle bone plate, with a longer shaft-anchor portion than in the embodiment of FIG. 10, constructed in accordance with aspects of the invention.

FIG. 14 is a side elevation (profile) view of the bone plate of FIG. 13.

FIG. 15 is a bottom view of the bone plate of FIG. 13.

FIG. 16 is a top plan view of an embodiment of a medial condyle bone plate for fixing medial-distal portions of a left or right humerus, particularly the medial condyle, constructed in accordance with aspects of the invention.

FIG. 17 is a side elevation (profile) view of the bone plate of FIG. 16.

FIG. 18 is a bottom view of the bone plate of FIG. 16.

FIG. 19 is a cross-sectional view of the bone plate of FIG. 16, viewed generally along line 19-19 of FIG. 16.

FIG. 20 is a cross-sectional view of the bone plate of FIG. 16, viewed generally along line 20-20 of FIG. 16.

FIG. 21 is a top plan view of another embodiment of a medial condyle bone plate, with a longer end-anchor portion than in the embodiment of FIG. 16, constructed in accordance with aspects of the invention.

FIG. 22 is a side elevation (profile) view of the bone plate of FIG. 21.

FIG. 23 is a top plan view of yet another embodiment of a medial condyle bone plate, with a longer end-anchor portion than in the embodiment of FIG. 21, constructed in accordance with aspects of the invention.

FIG. 24 is a side elevation (profile) view of the bone plate of FIG. 23.

FIG. 25 is a top plan view of still another embodiment of a medial condyle bone plate, with its end-anchor portion having a larger radius of curvature than in the embodiment of FIG. 23, in accordance with aspects of the invention.

FIG. 26 is a side elevation (profile) view of the bone plate of FIG. 25.

FIG. 27 is a top plan view of an embodiment of a posterior bone plate for fixing distal-posterior portions of a left or right humerus, particularly the lateral condyle and capitellum, constructed in accordance with aspects of the invention.

FIG. 28 is a side elevation (profile) view of the bone plate of FIG. 27.

FIG. 29 is a cross-sectional view of the bone plate of FIG. 27, viewed generally along line 29-29 of FIG. 27.

FIG. 30 is a cross-sectional view of the bone plate of FIG. 27, viewed generally along line 30-30 of FIG. 27.

FIG. 31 is a top plan view of an embodiment of an olecranon bone plate for fixing proximal-posterior portions of a left or right ulna, particularly the olecranon, constructed in accordance with aspects of the invention.

FIG. 32 is a side elevation (profile) view of the bone plate of FIG. 31.

FIG. 33 is a bottom view of the bone plate of FIG. 31.

FIG. 34 is a cross-sectional view of the bone plate of FIG. 31, viewed generally along line 34-34 of FIG. 32.

FIG. 35 is a cross-sectional view of the bone plate of FIG. 31, viewed generally along line 35-35 of FIG. 32.

FIG. 36 is a cross-sectional view of the bone plate of FIG. 31, viewed generally along line 36-36 of FIG. 32.

FIG. 37 is a top plan view of another embodiment of an olecranon bone plate, with longer shaft and end-anchor portions than in the embodiment of FIG. 31 but lacking prongs, constructed in accordance with aspects of the invention.

FIG. 38 is a side elevation (profile) view of the bone plate of FIG. 37.

FIG. 39 is a top plan view of yet another embodiment of an olecranon bone plate, with a longer shaft-anchor portion than in the embodiment of FIG. 31, constructed in accordance with aspects of the invention.

FIG. 40 is a side elevation (profile) view of the bone plate of FIG. 39.

FIG. 41 is a top plan view of still another embodiment of an olecranon bone plate, with a longer shaft-anchor portion than in the embodiment of FIG. 39 and configured for attachment to a left ulna, in accordance with aspects of the invention.

FIG. 42 is a side elevation (profile) view of the bone plate of FIG. 41.

FIG. 43 is a top plan view of an embodiment of a coronoid bone plate for fixing anterior-proximal portions of a left ulna, particularly the coronoid, in accordance with aspects of the invention.

FIG. 44 is a side elevation (profile) view of the bone plate of FIG. 43.

FIG. 45 is a bottom view of the bone plate of FIG. 43.

FIG. 46 is an end view of the bone plate of FIG. 43, viewed from the proximal end to a central region of the plate.

FIG. 47 is another end view of the bone plate of FIG. 43, viewed from the distal end to the central region of the plate.

FIG. 48 is a bottom plan view of another embodiment of a coronoid bone plate, with a longer shaft-anchor portion than in the embodiment of FIG. 43, in accordance with aspects of the invention.

### DETAILED DESCRIPTION

The invention provides sets of bone plates and components thereof for use in reducing and/or fixating bone discontinuities. The sets may be selected for use With particular bones or regions of bones, such as periarticular regions of an elbow, as described below. The bone plates may be configured to be easily identified, to be readily fitted to their intended targets, and/or to serve as a template for bone reduction, among others. The plates also may be configured to enhance stabilization of the targeted fractures.

Further aspect's Of the invention are described in the following sections: (I) bone-plate indicators, (II) target-defined structure, (III) sets of bone plates, (IV) bone-plate structure, (V) periarticular bone plates for the elbow, (VI) lateral condyle bone plates, (VII) medial condyle bone plates, (VIII) posterior humerus bone plates, (IX) olecranon bone plates, (X) coronoid bone plates, and (XI) exemplary uses of the periarticular elbow plates.

I. Bone Plate Indicators

The bone plates may be configured to be easily identified by using one or more indicators to identify (1) size, (2) handedness, (3) orientation, and/or (4) intended anatomical target region, among others. The indicators generally comprise any mechanism for distinguishing one bone plate from another, excluding mechanisms such as size or shape necessary for the function of the particular plate.

The bone plates may use color as an indicator, for example, by using different colors for different plates or portions thereof. Accordingly, plates with different colors may be intended for use on different anatomical target regions and/or a different side of the body (left/right). The colors may include red, blue, purple, green, silver, and/or gold, among others. The colors may be selected arbitrarily or according to preselected criteria, such as green for right and blue for left because green and right have five letters and blue and left have four letters, or green for right and red for left because green is used for starboard and red is used for port in nautical contexts. Plates configured for use on both the left and right sides of the body (nonhanded) may have the same color, but a color distinct from the colors used to indicate handedness. Alternatively, nonhanded plates may have colors that are different from the colors that indicate handedness and different from each other, to relate the intended anatomical targets of the plates. Plates may include two or more colors, for example, one color to indicate an anatomical target region of bone and another color to indicate handedness. The colors may be visible on one or plural surfaces of the bone plates. For example, the colors may be visible on the bone-facing surface, the bone-opposing surface, the sides, and/or regions thereof.

Alternatively, or in addition, the bone plates may use labels as an indicator, for example, by using different markings (writings, etchings, etc.) on different plates or portions thereof. The markings may include the name of a bone or a portion of a bone (e.g., condyle, olecranon, etc.) and/or the handedness of the bone (e.g., left, right, etc.), among others. Indicators may be selected so that they do not interfere with the function of the corresponding plate, for example, by adversely affecting its size, shape, strength, and/or biocompatibility.

II. Target-Defined Structure

The bone plates also may be configured to be easily fitted to their intended target. For example, the bone plates may be sized and/or precontoured (i.e., prebent, cast, machined, etc.) to a shape that at least nearly matches a particular region of bone, so that the surgeon needs to adjust the shape only slightly before application in some cases, and not at all in other cases. This precontouring (or preshaping) also may reduce or eliminate the degree to which a fracture must be fixed before applying the plate, since matching the bone and plate will help to fix the fracture.

The plates may be precontoured in various ways. The plates may be precontoured in two or three dimensions to wrap around the intended region of bone. Moreover, these plates may be configured so that each successive plate includes an additional precontoured portion configured to wrap around an additional portion of the intended target region of bone, for example a shaft region and/or periarticular region, among others. In some cases, bone plates may be somewhat undercontoured so that some additional bending is required to match the bone plate to the contour of bone. Alternatively, or in addition, the bone plates may possess a handedness necessary to fit a left or a right bone.

Further aspects of precontouring, such as plate handedness and/or three-dimensional structure, are described below in Sections V to X.

III. Sets of Bone Plates

The bone plates or sets of bone plates may be accompanied by various ancillary materials including instructions, fasteners, and a case, among others. The instructions may include a description of how the plates may be used, relationships between colors and target anatomical region, additional medical indications, and so on. The fasteners may include any device capable of affixing the plate to a bone, such as bone screws, wires, and so on. The case may include a protective covering and interior compartments for separating bone plates, fasteners, and so on. The case May facilitate use by organizing materials, so that they may be located and identified quickly during use.

Precontoured (or preformed) sets of bone plates may be provided for any suitable periarticular and/or anatomical regions or set of regions. The plates may be sold collectively, in any combination, selected, for example, for a particular bone, region of bone, size of bone, and so on. For example, a set of bone plates may be configured for use on periarticular, shaft, plate, junction, and/or interarticular regions of an elbow, knee, shoulder, hip, wrist, ankle, skull, vertebral column, arm, leg, hand, foot, pelvis, and/or the like. The bone plates may be sold and used individually and/or collectively. The plates may be sold independently, for certain applications, or to replace plates used from a kit.

IV. Bone-Plate Structure

The plates may be of a sturdy yet malleable construction. Generally, the plates should be stiffer and stronger than the section of bone spanned by the plate, yet springy enough not to strain the bone significantly. Suitable materials include titanium, stainless steel, and/or other biocompatible materials.

The plates may be configured to reduce irritation to the bone and surrounding tissue. For example, the plate may be formed of a biocompatible material, as described above. In addition, the plate may have a low and/or feathered profile to reduce its protrusion into adjacent tissue and rounded, burr-free surfaces to reduce the effects of such protrusion.

The plates may be sized to conform to particular regions of bone, or to different portions of the same region of bone, among others. The plates are generally elongate (at least before bending), with a length L, a width W, and a thickness T. Here, length L>width W>thickness T. In use, the long axis of the elongate plates may be aligned with the long axis of the corresponding bone or may extend obliquely relative to the long axis, for example, as in some of the coronoid plates described below in Section X. The length and/or width of the plates may be varied according to the intended use, for example, to match the plate with preselected region of bone. The terms “in profile” or “profile” will be used throughout to refer to a side view of a bone plate, generally parallel to an axis that defines a width of the plate.

The thickness of the plates is generally defined by a distance between inner (facing bone) and outer (opposing bone) surfaces of the plates. The thickness of the plates may be varied according to the intended use, for example, to make the plate thinner as it extends over protrusions (such as processes, condyles, tuberosities, and/or the like), reducing its profile and/or rigidity, among others. The thickness of the plates also may be varied to facilitate use, for example, to make the plate thinner where it typically needs to be contoured to facilitate bending. In this way, the plate may be thicker and thus stronger in regions where it typically does not need to be contoured, generally along the shaft of the bone.

The plates and their surfaces also may be shaped to conform to particular anatomical regions of bone, on the same bone or different bones, among others. In particular, the plates may be preshaped, that is, precontoured (preformed), generally to fit an average target anatomy, for example, a population-averaged shape of a particular anatomical region. The average anatomy may be a human (or other animal) anatomy averaged over any suitable set, for example, adults, adult males, adult females, people that fall within a particular size range, children of a given age, and/or so on. The preshaping allows the inner or bone-facing surface of the plate to follow and substantially match the three-dimensional contour of a bone, along the length of the plate and/or across the width of the plate. For example, the plates may include curved, bent, twisted, and/or tubular inner surfaces that are adapted to face bone and to guide the plates to set onto the bones, initially to enhance fixation and/or to template reduction of bone, and subsequently to increase stability, by grabbing and holding bone fragments. In some embodiments, the plates may be somewhat undercontoured along their long axes, for example, to accommodate soft tissue between a portion of the plate and the bone, or to allow additional custom contouring pre- or peri-operatively, among others.

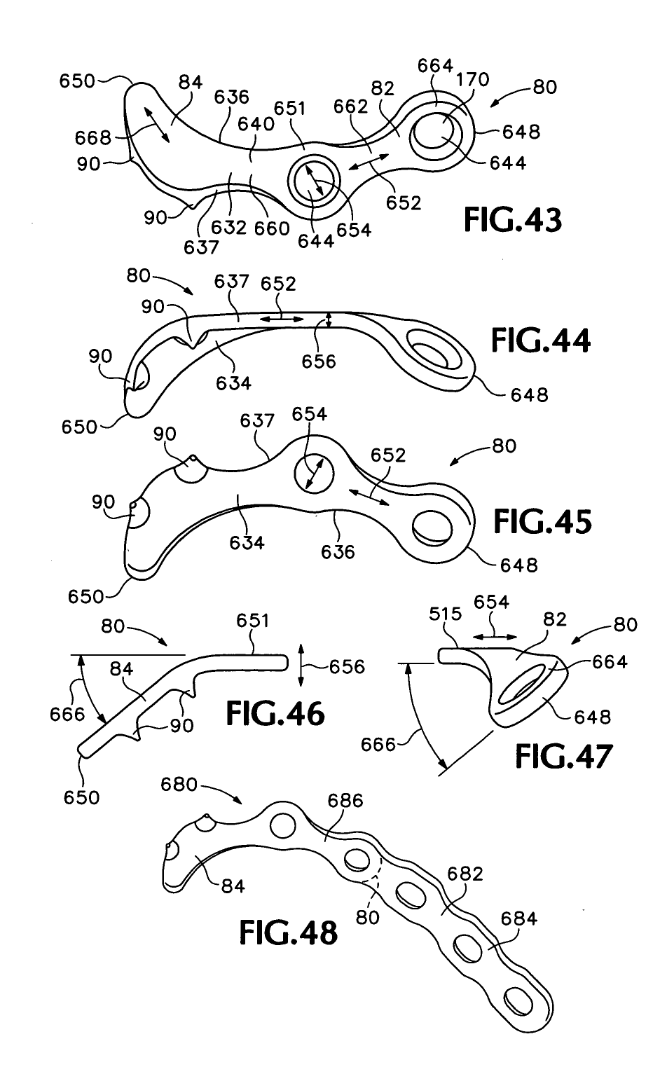

The plates also may include spacing members, such as prongs or other projections. Spacing members may be configured to project generally orthogonal to a proximal surface of bone, when the plates are attached in their intended orientation to bone. Accordingly, spacing members may project from the sides and/or bone-facing surfaces of bone plates in a substantially orthogonal direction relative to a plane defined locally by length and width of a bone plate. Spacing members such as broad prongs may be used to position at least a portion of the plate away from the bone, so that tendons, and possibly nerves, blood vessels, and the like, may pass under the plate without being pinched or damaged. Alternatively, or in addition, spacing members such as narrow and/or sharp prongs may be used to grasp the bone for increased fixation, in conjunction with and/or independent of additional fixation mechanisms. For example, prongs and screws are used in close apposition in the olecranon plates in Section IX, whereas prongs and screws are used far apart in the coronoid plates in Section X (FIGS. 43-48). Moreover, in the coronoid plates, prongs serve as the primary or exclusive fixation mechanism on one end of the plate, and screws serve as the primary or exclusive fixation mechanism on the other end of the plate.

The plates may include at least one, and preferably two, anchor portions configured to receive fasteners to attach the plate to the bone. In some embodiments, such as those described below, an anchor portion may be configured distinctly to attach the plate to distinct regions of a bone. For example the anchor portion may be configured for fixation to a diaphyseal (shaft) portion using a plurality of bone screws. The shaft portion generally includes all central portions of a long bone and may give the long bone strength and largely defines its length. Alternatively, or in addition, the anchor portion may be configured for stabilization of a metaphyseal (end) portion of a bone. The end portion may include periarticular structures (such as processes, fossae, cavities, condyles, projections, tuberosities, and/or the like) for limiting, defining, protecting, or enabling articulation, among others. In some cases, such as the coronoid plates described below in Section X, an anchor portion at an end of a plate may be replaced with a buttress portion that stabilizes periarticular bone using spacing members or projections rather than by attachment with fasteners.

The plates also may include a bridge or intermediate portion configured to join flanking anchor portions and/or to bridge the discontinuity in the bone. The bridge portion may have an altered flexibility, thickness, and/or width relative to the flanking anchor portions, for example, an increased flexibility to promote bending and/or twisting two flanking anchor portions relative to each other. The bending and/or twisting may be carried out during manufacture of the bone plates and/or during use, as described further below. The bridge portion may include openings (see below) or may be free of openings. The bridge and anchor portions may be defined statically (e.g., by the plate contour and/or the positions of the anchoring sites for fasteners) and/or dynamically (e.g., by the position of the discontinuity relative to portions of the plate).

The plates generally include a plurality of apertures or openings adapted to perform different functions. The openings may be adapted to receive fasteners for affixing the plates to the bone. Alternatively, or in addition, the openings may be adapted to alter the local rigidity of the plate and/or to facilitate blood flow to the fracture to promote healing.

The openings May have a variety of geometries and dimensions. For example, some openings may be elongate (such as substantially oval, among others), whereas Other openings may be substantially circular. The elongate openings may be used as reduction slots, allowing the plate to slide back and forth along the long axis of the opening for final positioning of the plate after a fastener is affixed to the bone through the opening. Alternatively, or in addition, the elongate openings may allow greater flexibility in the angle of insertion of a fastener. By contrast, the circular openings may be used for attaching an anchor portion of the plate to bone that has been positioned finally relative to the anchor portion. For example, circular openings may be included in a shaft-anchor portion of a plate for use in placing additional fasteners into a bone shaft after the plate is finally positioned relative to the shaft. Alternatively, or in addition, circular openings may be included in an end-anchor portion of a plate for use in placing fasteners into periarticular bone that is (or is being) finally positioned relative to the end-anchor portion of the plate. Fasteners may be placed into bone using circular openings in the end-anchor portion of a plate before and/or after final positioning of the corresponding shaft-anchor portion relative to the shaft. The openings may include counterbores that allow fasteners to lie substantially flush with the top surface of the plates. Moreover, the openings (particularly the elongate openings) may include tapered counterbores that bias a fastener toward (or away from) a bone discontinuity, for example, to provide compression.

The openings may have various sizes, depending on their intended usage. For example, if used with fasteners, the openings may be sized to receive and effectively hold fasteners of different size, such as number 2.7, 3.5, and/or 4.0 bone screws, in order of increasing size. Generally, the smaller the opening, the smaller the screw, so that smaller openings allow relatively larger numbers of screws to be used with a given plate. Generally, also, the larger the plate, the larger the number of openings, so that larger plates allow relatively larger numbers of screws to be used. The openings may have a hybrid arrangement, such as a size 3.5 in the shaft-anchor portion and a size 2.7 in the end-anchor portion.

The openings also may have any suitable positions or densities within each anchor portion of a the plate. The openings may be positioned along a middle axis of the plate, with the center of each opening centered across the width at each position. Alternatively, one or more of the openings may be disposed off-center, that is, disposed asymmetrically or laterally relative to the local width of the bone plate. For example, some or all openings may be staggered in position, such as alternatively disposed at lateral positions on opposing sides of the middle axis. Alternatively, or in addition, two or more of the openings may be aligned side-by-side (transversely), as described below in Section IX for the olecranon plates. Openings that precede and/or follow the laterally disposed openings or side-by-side openings may be positioned along the middle axis of the bone plates, increasing the density of screws that may be used. Spacing between openings (center-to-center or side-to-side) may be constant or varied. For example, some or all of the openings of the end-anchor portion may be clustered together at a higher density to increase the number of screws that can be used to fix (stabilize) the associated segment(s) of bone(s) via the plate. The openings may be positioned at different positions on a cured plate so that the screws interact with the bone and/or each other in a three-dimensional pattern, in some cases interdigitating or locking together so that the screws are fixed to more than just bone. In these situations, it may be preferable to use tapered screws that can pass one another or deflect or bend off one another rather than hit up against one another and stop. The openings also may be positioned so that screws will project along a long axis of the bone, rather than a transverse axis, increasing the length of screw and the number of threads that contact the bone, and thus increasing purchase.

The fasteners generally comprise any mechanism for affixing a bone plate to a bone, including screws and wires, among others. A preferred fastener is a bone screw, as mentioned above, including unicortical, bicortical, and/or cancellous bone screws. Unicortical and bicortical bone screws typically have relatively small threads for use in hard bone, such as with the shaft portion of a bone, whereas cancellous bone screws typically have relatively larger threads for use in soft bone, such as near the ends (periarticular regions) of a long bone. Unicortical bone screws penetrate the bone cortex once, adjacent the bone plate. Bicortical bone screws penetrate the bone cortex twice, adjacent the bone plate and opposite the bone plate. Generally, unicortical screws provide less support than bicortical screws, because they penetrate less cortex. The size and shape of the fasteners may be selected based on the size and shape of the openings, or vice versa, as described above. Bone screws are particularly preferred for use in fixating the shaft-anchor portion of a bone plate, whereas various fasteners may be used to fixate and stabilize bone with the end-anchor portion. A preferred fastener for each portion is an Acumed bone screw having a screw head adapted to fit the plate construction.

V. Periarticular Plates for the Elbow

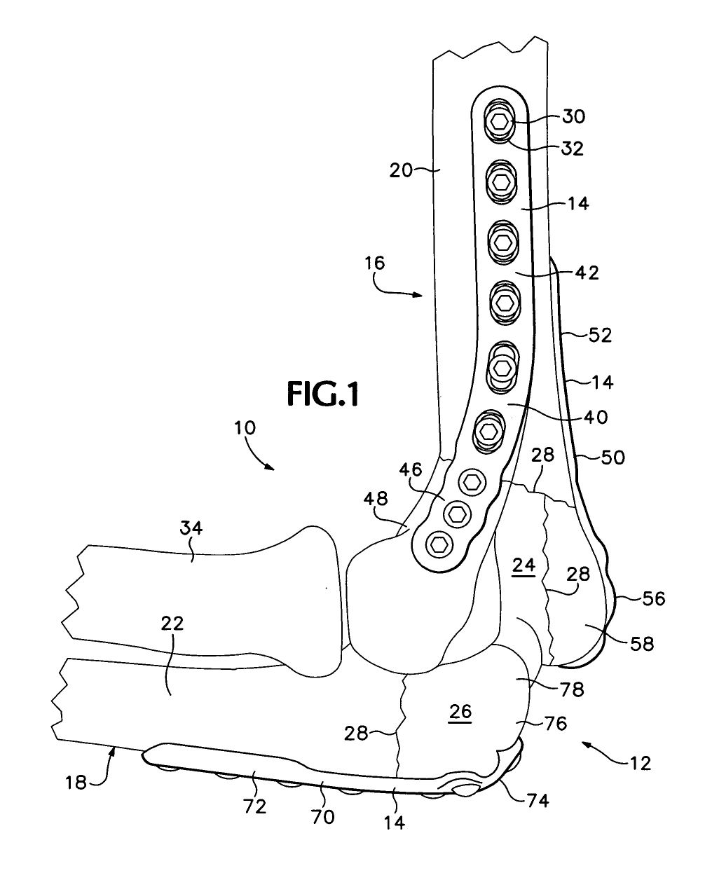

This section introduces a set of precontoured bone plates that may be used to fix bone discontinuities within the periarticular region of an elbow; see FIGS. 1-3. Throughout this description, the bone plates have been assigned relative sizes to provide a nomenclature that assists in describing the plates. These sizes are intended to improve clarity of the description and are not intended to define or limit the scope of the invention. In particular, other plates that are smaller, larger, or intermediate in size relative to the plates that are described and shown, and that have fewer or more openings, are within the scope of the invention.

The humerus is the only bone in the upper arm. The humerus includes a proximal region closest to the body that articulates with the glenoid fossa of the scapula and a distal region farthest from the body that articulates with corresponding portions of the ulna and radius. The distal humerus includes a variety of regions. The medial and lateral columns represent the structural transitions linking the elbow joint and the humeral shaft. The medial and lateral condyles are projections from the respective columns of the distal humerus. The capitellum is the lateral convex portion of the distal condyles. It articulates with the radius. The trochlea is the more medial, spool-shaped section of the distal condyles that articulates with the ulna. The coronoid fossa is a small depression on the anterior surface above the trochlea that receives the coronoid of the ulna when the elbow is flexed (bent). The olecranon fossa is a deep depression on the posterior surface above the trochlea. It receives the olecranon of the ulna when the elbow is extended (straightened).

The ulna and radius are the only bones in the forearm, where the ulna is medial and the radius is lateral. These bones include proximal regions that articulate with the distal portion of the humerus, as described above. These bones also include distal regions that articulate with the various bones of the wrist. The proximal ulna includes a variety of regions, including the olecranon posteriorly and the coronoid anteriorly, which interact with the humerus, as described above. The proximal ulna also includes the trochlear (or semilunar) notch, a smooth articular concave surface that lies on the anterior surface of the olecranon and extends onto the articular surface of the coronoid.

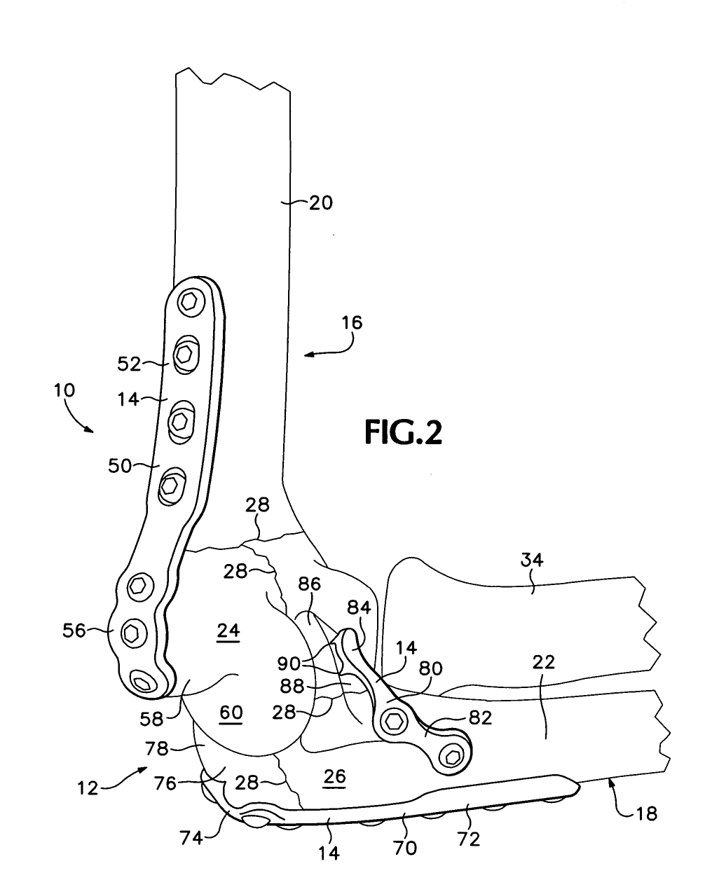

FIGS. 1 and 2 show the bones of a left elbow region 10 fixed with exemplary members of a set 12 of precontoured bone plates 14. Left elbow region 10, shown here in a flexed configuration with the hand pronated (not shown), includes the distal humerus 16 and proximal ulna 18. The distal humerus and proximal ulna have shaft regions 20, 22 and periarticular (end) regions 24, 26, respectively. Each bone plate 14 spans a bone discontinuity 28, such as a fracture, osteotomy, and/or the like. In addition, each bone plate is attached with fasteners, in this case bone screws 30, which extend through openings or apertures 32 in plates 14 and into bone. Here, the proximal radius 34 does not carry a bone plate.

A lateral condyle plate 40 is attached to distal humerus 16. Plate 40 includes a shaft-anchor (or proximal) portion 42 fixated to a lateral portion of shaft region 20, and an end-anchor (or distal) portion 46 attached to and stabilizing a lateral portion of periarticular region 24, particularly lateral condyle 48. Further aspects of lateral condyle plates are described below in Section VI.

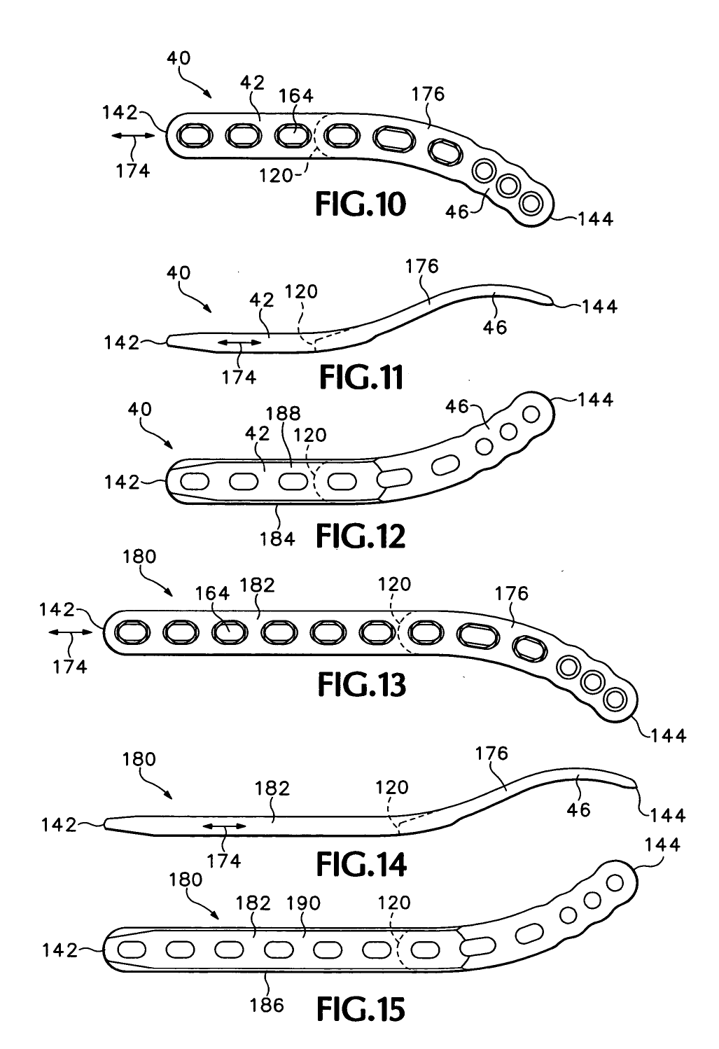

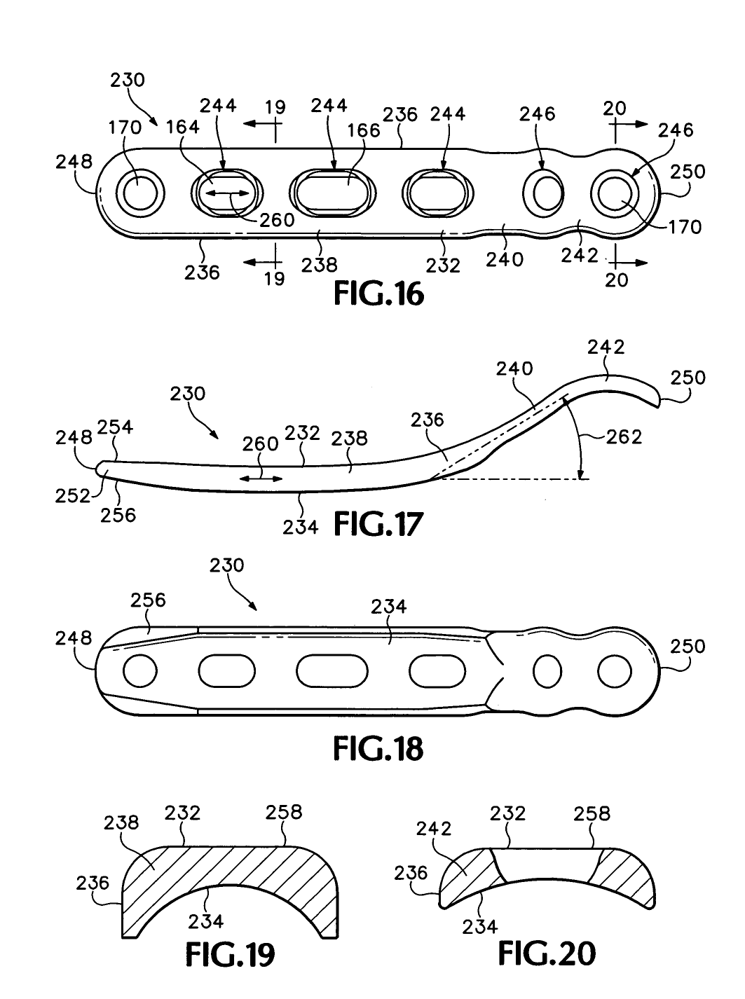

A medial condyle plate 50 also is attached to the distal humerus 16. Plate 50 includes a shaft-anchor (or proximal) portion 52 that is attached to a medial portion of periarticular region 24, and an end-anchor (or distal) portion 56 that is attached to medial condyle 58. Medial condyle plate 50 is a medium-sized embodiment that extends to an intermediate position along the proximal-distal axis of medial condyle 58. Smaller embodiments may terminate, for example, at a more proximal position along medial condyle 58 (see FIGS. 16-20). By contrast, larger embodiments may extend farther distally over medial condyle 58, for example, attaching at a distal portion to a medial surface of the trochlea 60 (see FIGS. 23-26). Further aspects of medial condyle plates are described below in Section VII.

FIGS. 1 and 2 also show an olecranon plate 70 attached to proximal ulna 18. Plate 70 includes a shaft-anchor (or distal) portion 72 attached to proximal shaft region 22. Plate 70 also includes an end-anchor (or proximal) portion 74 attached to a posterior side of periarticular region 26, particularly olecranon 76. As shown here, olecranon plates may be configured so that the shaft-anchor portion is attached to a posterior surface of shaft region 22, so that the bone-facing surface of the plates is oriented generally orthogonal to the anterior-posterior axis of the proximal ulna.

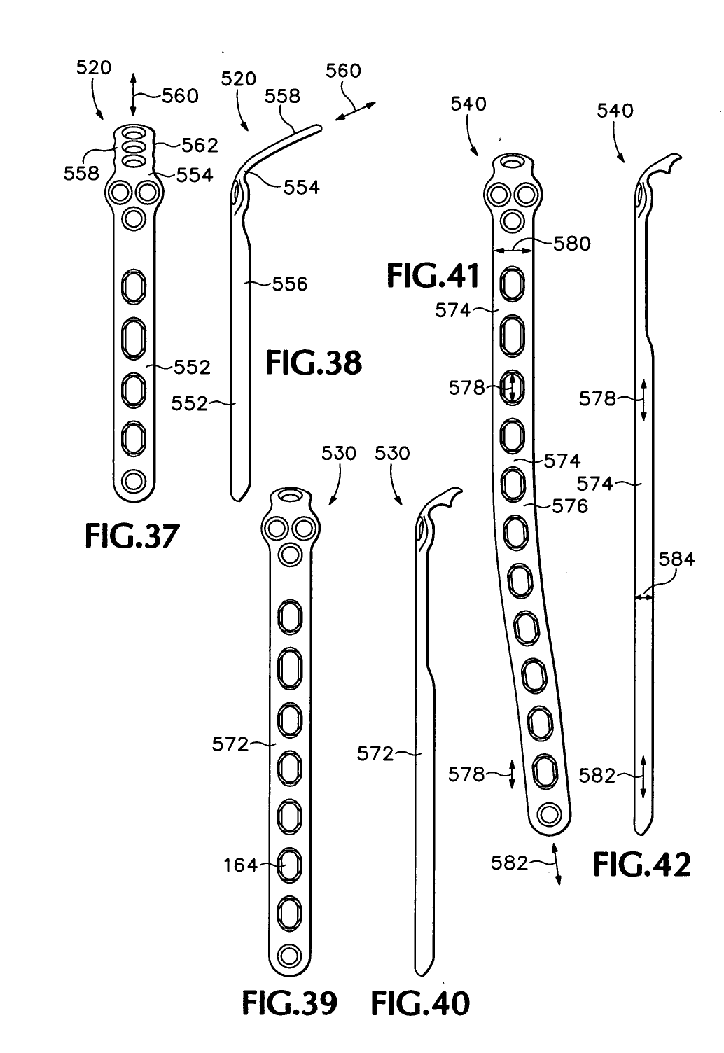

Olecranon plates may have any suitable size. Plate 70 is a smaller embodiment of an olecranon plate. In larger embodiments, the shaft-anchor portion may be configured to extend farther distally along shaft region 22 (see FIGS. 37-42), and/or the end-anchor portion may be configured to extend farther toward proximal tip 78 of olecranon 76 (see FIGS. 37-38), among others. Further aspects of olecranon plates are described below in Section IX.

FIG. 2 shows a coronoid plate 80 attached to proximal ulna 18. Plate 80 includes a shaft-anchor (or distal) portion 82 that attaches to the medial side of shaft region 22. Coronoid plate 80 also includes a buttress portion 84 that stabilizes coronoid 86 primarily through contact with a distally facing surface 88 of coronoid 86, for example, using prongs 90. In larger embodiments, coronoid plate 80 may extend, for example, farther distally along the medial side of shaft region 22. Further aspects of coronoid plates are described below in Section X.

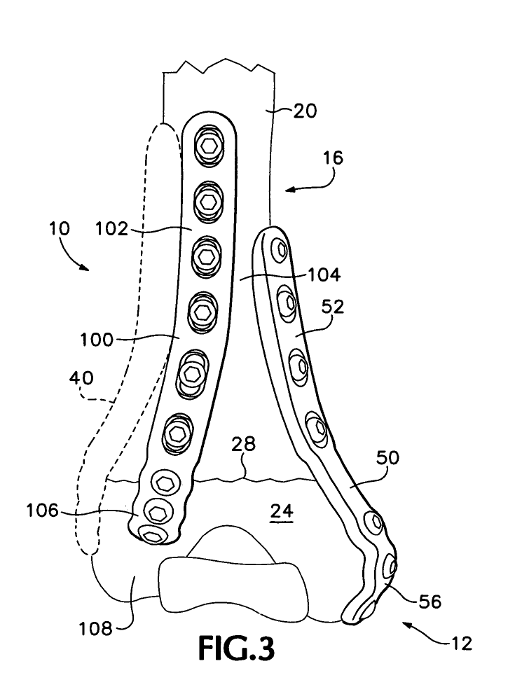

FIG. 3 shows an alternative fixation strategy using a posterior plate 100 in lieu of lateral condyle plate 40 (shown as dashed) on periarticular region 24 of distal humerus 16. In this strategy, posterior plate 100 and medial condyle plate 50 are disposed in a generally orthogonal arrangement, rather than the generally opposing or parallel relationship of lateral and medial plates 40, 50. Accordingly, posterior plate 100 includes a shaft-anchor (or proximal) portion 102 that attaches to a posterior side 104 of shaft region 20. In addition, posterior plate 100 includes an end-anchor (or distal) portion 106 that attaches to a posterior-lateral side of periarticular region 24, including capitellum 108. Further aspects of posterior plates are described below in Section VII.

VI. Lateral Condyle Bone Plates

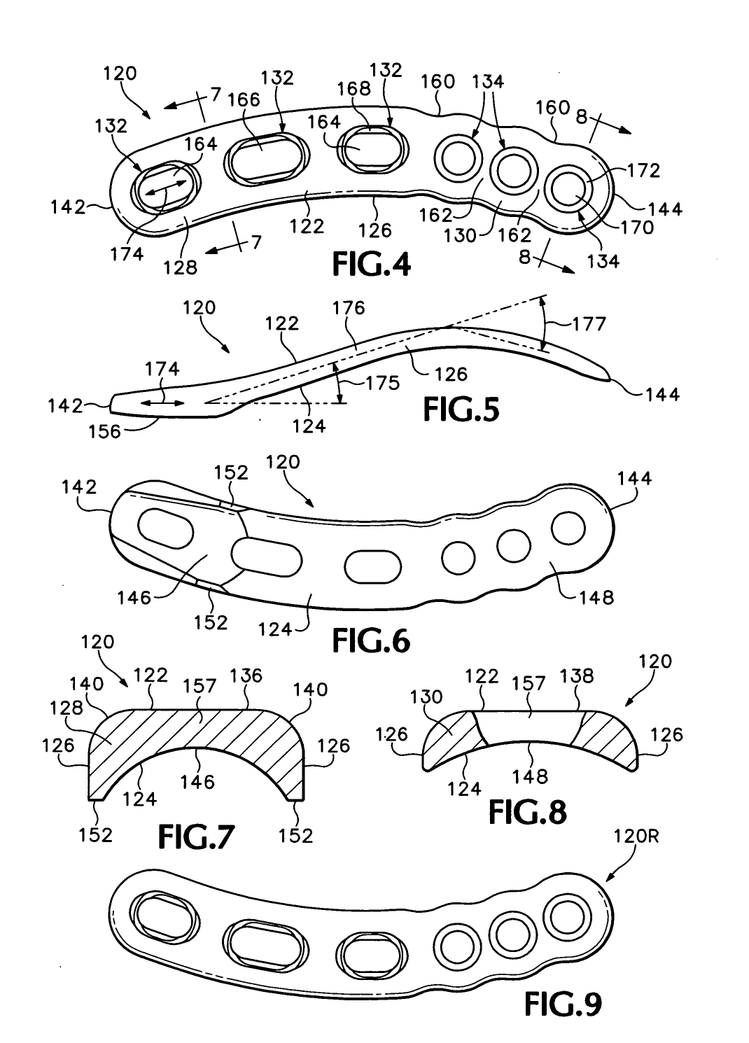

This section describes lateral condyle plates configured for fixing fractures of periarticular and/or shaft regions of the left and/or right distal humerus, particularly the lateral condyles; see FIGS. 4-15. Many of the features or aspects of the lateral condyle plates described herein also may be suitable for the other plates described above and in Sections VII to X below, and vice versa.

FIGS. 4-8 show top, side, bottom, and two cross-sectional views, respectively, of a smaller-sized lateral condyle plate 120. Plate 120 includes an outer (or bone-opposing) surface 122, an inner (or bone-facing) surface 124, sides 126, proximal (or shaft-anchor) portion 128, and distal (or end-anchor) portion 130. Proximal and distal sets of openings 132, 134 are defined by openings in proximal and distal portions 128, 130, respectively.

Outer surface 122 is configured to face away from bone when plate 120 is attached. FIGS. 7 and 8 show that outer surface 122 may be generally convex and/or linear in transverse cross section, for example, having a centrally disposed linear region, as shown at 136 and 138. Accordingly, regions of proximal and/or distal portions 128, 130 may be substantially planar on outer surface 122. Rounded or chamfered corners 140 may join sides 126 to top surface 122. Rounded corners 140 also may join one or both of proximal end 142 and distal end 144 (see FIG. 4) to outer surface 122, providing a generally rounded perimeter.

FIGS. 7 and 8 also show inner surface 124, which is configured to face toward bone. Accordingly, inner surface 124 may be generally concave, for example, having tubular surfaces 146, 148 in different portions that may vary in radius of curvature, as measured transversely. Here, tubular surface 146, disposed near proximal end 142, has a smaller radius of curvature than tubular surface 148 in distal portion 130. Tubular surfaces may be flanked by longitudinal chamfers (see FIGS. 6 and 7). For example, tubular surface 146 is flanked here by chamfers 152 that form a flattened region on inner surface 124 near proximal end 142.

The thickness of lateral condyle plate 120 may vary along the length and/or across the width of plate 120. FIGS. 7 and 8 show that plate 120 thins between proximal portion 128 and distal portion 130, so that the average thickness of the proximal portion is greater than the average thickness of the distal portion. (Average thickness (or width) is determined without considering thinning of proximal end and/or distal end.) Thinning may occur along sides 126 and/or at positions centered between sides 126. Thinning may occur as a gradual taper from proximal to distal portions 128, 130 or at one or more fairly discrete positions along the length of the plate. A thinner distal portion may, for example, locally decrease the profile of the bone plate to minimize irritation. Alternatively, or in addition, proximal portion 128 may thin proximally near proximal end 142, to produce a tapered region 156 (see FIG. 5). Tapered region 156 may be produced, for example, by angled chamfers on outer surface 122 and/or inner surface 124, among others. Tapered region 156 may be useful, for example, to facilitate sliding proximal end 142 of plate 120 under soft tissue during positioning of the plate on bone. Alternatively, or in addition, thinning may occur transversely, along axes corresponding to width. For example, as shown in FIGS. 7 and 8, plate 120 thins or tapers towards center positions 157 from sides 126. In some embodiments, plate 120 may taper from center positions 157 toward sides 126. Transverse thinning, either thinning toward the sides and/or toward the center may occur in proximal and/or distal portions of the plate.

The width of lateral condyle plate 120, as measured between opposing sides 126, may vary along the length of the plate. In some embodiments, plate 120 narrows between proximal and distal portions 128, 130, so that the average width of proximal portion 128 is greater than the average width of distal portion 130. (Proximal and distal ends are not considered in calculating average width.) For example, plate 120 may include one or more narrowed regions 160, here in the form of scallops, at a position intermediate one or more pairs of openings of set 134 along the length of plate 120. Narrowed regions 160 are an example of a structure that may provide localized regions of decreased rigidity, for example, to direct bending pre- or peri-operatively, among others. The localized regions may be restricted along the length of proximal portion 130 to zones 162 disposed intermediate the openings. In some embodiments regions 160 may be narrowed relative to other regions of distal portion 130, but not relative to proximal portion 128. Alternatively, or in addition, decreased rigidity may be provided locally, for example, between openings, among others, by altering the thickness of the plate at zones 162.

Openings may vary in structure according to position within plate 120. Some or all openings of set 132 within proximal portion 128 may be elongate. Elongate openings may have the same length or differing lengths. Here, openings 164 are shorter than opening 166. Longer opening 166 may be disposed at any suitable position relative to shorter opening 164, for example, being the second elongate opening from the distal portion. Openings 164, 166 may function in compression, and thus may have beveled perimeters or counterbores 168 that produce a ramping action. The ramping action applies a horizontal force on the plate that is parallel to the long axis of the opening as a fastener is tightened in the opening. When elongate openings of different lengths are included, such a's openings 164 and 166, stepped (sequential) reduction or compression of bone may be produced by first tightening a bone screw in opening 166 and then tightening a bone screw in one of the flanking elongate openings 164. By contrast, some or all of openings 134 defined by distal portion may be fixed-position, or circular openings 170, which generally include counterbores 172. Accordingly, as shown here, elongate openings 164, 166 and circular openings 170 may be substantially or completely segregated according to position within plate 120 and within other bone plates described herein.

Openings may have a spacing or density that changes in different portions or sub-portions of the bone plate. Generally, openings are disposed at a higher density (openings per length of plate) in the distal portion of the plate. For example, plate 120 includes a plurality of circular openings 170 in distal portion 130 that are more closely spaced (center-to-center) and thus have a higher density than the elongate openings 164, 166 in proximal portion 128.

Lateral condyle plate 120 may be bent or shaped along its length so that bone-facing surface 124 at least substantially matches a lateral region of the distal humerus. For example, when viewed from orthogonal outer surface 122 as in FIG. 4, plate 120 defines an arc that bends rightward from long axis 174, defined near proximal end 142, as the plate extends from proximal to distal ends 142, 144. The arc may extend near distal end 144 at an angle of about 20 to 40 degrees, or about 30 degrees relative to long axis 174. The arc may dispose distal portion 130 more anteriorly on the distal humerus than proximal portion 128, when plate 120 is attached as intended (see FIG. 1). When viewed in profile, with distal end 144 on the right (as in FIG. 5), plate 120 bends upward (counterclockwise) from long axis 174 by an angle 175 of about 10 to 30 degrees, about 15-25 degrees, or about 20 degrees, to form a bridge (or intermediate) portion 176 and a concave bend along the length and with respect to outer surface 122. Bridge portion 176 may be generally similar in size or longer than distal portion 130, for example, having a length relative to distal portion 130 of about 3:1 to 0.5:1, or about 2:1 to 1:1. Bridge portion 176 may include one or more elongate openings as shown. In profile, distal portion may bend downward (clockwise) from a long axis defined by bridge portion 176 (shown dashed), to define a convex bend along the length and with respect to outer surface 122, and defining an angle 177 that is at least equal to, or about twice as great as, angle 175, or about 20 to 60 degrees, about 30 to 50 degrees, or about 40 degrees. Distal portion 130 may have a length of about 1 to 3 centimeters, or about 2 centimeters.

FIG. 9 shows a top view of a right-handed embodiment of left-handed plate 120, lateral condyle plate 120R. Plate 120R is configured for fixing bone discontinuities in the distal-lateral right humerus of a body. Thus plate 120R arcs to the left, rather than the right as described above for plate 120. Plates 120 and 120R, and all other left- and right-handed plates described herein, may be related to each other by substantial or complete mirror-image symmetry. Accordingly, only the left-handed embodiment of each handed pair of bone plates is shown and described below. In alternative embodiments, lateral condyle plates may be configured to lack handedness, that is, configured to fix fractures of both the left and right distal humerus.

FIGS. 10-15 show top, side, and bottom views, respectively, of exemplary embodiments of intermediate-sized (FIGS. 10-12) and larger-sized (FIGS. 13-15) lateral condyle bone plates 40, 180 (see also FIGS. 1 and 2 for plate 40). Each of plates 40, 180 is a left-handed embodiment for use on a distal left humerus. These plates exemplify how any of the bone plates described herein may be modified by extending (or truncating) the proximal and/or distal portions to fit over more (Or less) contiguous bone. Plates 40 and 180 may be considered to be elongated derivatives of plate 120, Whose proximal extent is shown in dotted outline and labeled. In these plates proximal portion 128 of plate 120 has been extended linearly to produce proximal portions 42, 182, respectively. Linear extension may elongate chamfers 152 (see FIG. 6) to produce chamfers 184, 186, may increase the number of elongate openings 164 or 166 (or circular openings) to maintain an approximately constant density of openings and/or may extend tubular surface 146 to produce surfaces 188, 190. In other embodiments, any suitable extension may be selected.

Plates 40, 180 may have three-dimensional structures and contours produced by adding a linear extension proximal portion 128 of plate 120. When viewed from the outer surface, as in FIGS. 10 and 13, each of plates 40 and 180 extends linearly from proximal end 142 to bridge portion 176. At bridge portion 176, each plate bends upward and then bends downward to transition to distal portion, as described above for plate 120. Accordingly, when viewed in profile, as in FIGS. 11 and 14, each of plates 40, 180 has a concave bend followed by a convex bend with respect to the outer surface and along the length from proximal end 142 to distal end 144. Each of the proximal, bridge, and distal portions may be generally linear in profile adjacent the concave and/or convex bends, as shown here, or may be arcuate.

VII. Medial Condyle Bone Plates

This section describes medial condyle plates configured for fixing fractures of periarticular and/or shaft regions of the left and/or right distal humerus, particularly the medial condyles; see FIGS. 16-26.

FIGS. 16-20 show top, side, bottom, and two cross-sectional views, respectively, of a smaller-sized medial condyle plate 230. Plate 230 includes an outer (or bone-opposing) surface 232, an inner (or bone-facing surface) 234, and sides 236. Proximal (or shaft-anchor) portion 238 extends to join bridge (or intermediate) portion 240, which in turn joins distal (or end-anchor) portion 242. Proximal and distal portions define sets of openings 244, 246, and extend in generally opposite directions to proximal and distal ends 248, 250.

Medial condyle plates in general, and plate 230 in particular, may include any suitable features described above for lateral condyle plates 40, 120, 200, or described elsewhere in this description for other bone plates. For example, medial condyle plate 230 may include a tapered region 252 at proximal end 248 (see FIGS. 17 and 18). Tapered region 252 may be formed by top and/or bottom chamfers 254, 256, similar to tapered region 156 of plate 120. Plate 230 may include cross-sectional configurations in the proximal and distal portions, shown in FIGS. 19 and 20, that are similar to those of lateral condyle plates described above. Accordingly, plate 230 many thin distally or transversely, for example, tapering centrally in transverse cross section. Alternatively, or in addition, plate 230 in transverse cross section may include distinct radii of curvature in proximal and distal portions 238 and 242 on inner surface 234, for example, having a larger radius in distal portion. Plate 230 in transverse cross section may have linear regions 258 along outer surface 232. Plate 230 may vary in width, for example, narrowing in bridge portion 240, distal portion 242 and/or having oscillating width as described above for plate 120.

Opening sets 244 and 246 may have any of the features described above for opening sets 132, 134 of plate 120. Accordingly, opening sets 244, 246 may be spatially segregated according to type, with elongate openings disposed in proximal portion 238, including openings 164, 166 of different length as shown. However, plate 230 and any of the other plates described herein may have openings of three or more lengths in proximal portion 238. For example, plate 230 also has a circular opening 170 disposed at proximal end 248. Bridge portion 240 may be opening-free. Distal portion 242 may plural circular openings 170 and may or may not include elongate openings.

Medial condyle plates may be nonhanded, that is, configured to be attached to medial periarticular and/or shaft regions of both the left and right distal humerus. Accordingly, plate 230 and other nonhanded medial condyle plates may be substantially or completely symmetrical bilaterally, that is, plate 230 may have substantial or complete mirror-image symmetry across a plane that is centered between sides 236 and generally orthogonal to axes that parallel width. The symmetry may relate to opening placement and/or the plate perimeter. Thus, plate 230 may appear to be generally linear when viewed along a line that is orthogonal to a plane defined by length and width, as in FIGS. 16 and 18. In alternative embodiments, medial condyle plates may be configured to have handedness, that is, configured to fix fractures of either the left or right distal humerus (but not both).

Plate 230 may be contoured to have a partially or completely nonlinear profile when viewed in profile, as in FIG. 17. Specifically, plate 230 may have a linear profile proximally, extending along long axis 260 from proximal end 248 centrally. Plate 230 then may bend upward, more centrally along the length, for example, to define a concave arc With respect to outer surface 232 and the length, as the plate extends to bridge region 240. As a result, bridge portion 240 distally may define an angle 262 relative to long axis 260 of about 20 degrees to 50 degrees, about 25 degrees to about 45 degrees, or about 35 degrees. Distal portion 242 may bend downward, toward inner surface 234, to define a convex arc (relative to outer surface 232) in distal portion 242. The concave arc disposed more proximally may have a radius that is at least twice the radius of the convex arc. The convex arc may have a radius of about 0.4 to 2 cm, about 0.5 to 1.5 cm, or about 0.8 cm.

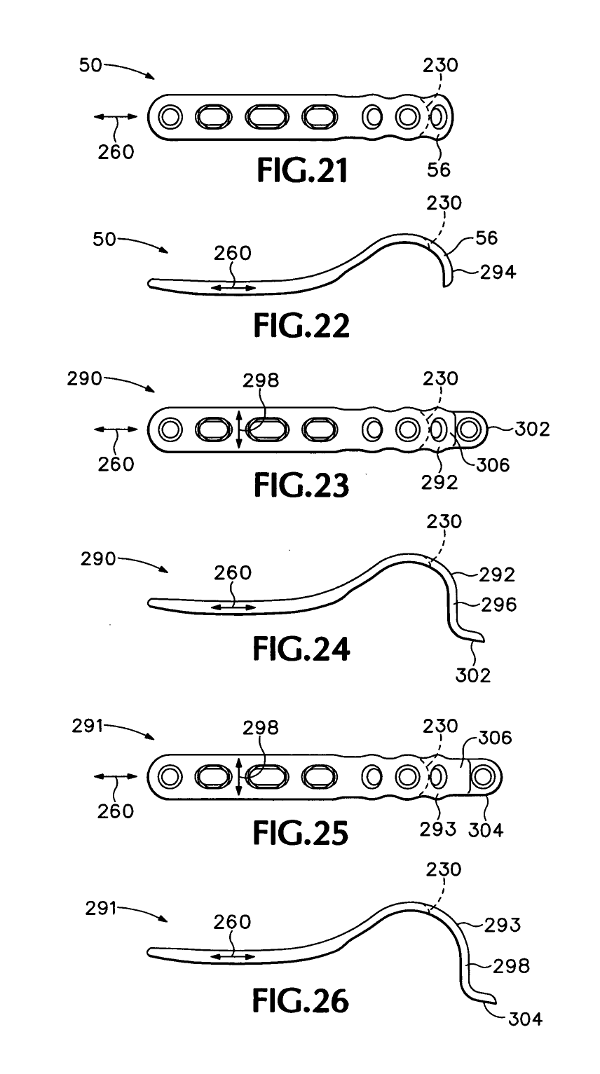

FIGS. 21-26 show top and side views for three alternative embodiments of medial condyle plates, plates 50, 290, and 291, respectively (see also FIGS. 1 and 2 for plate 50). Each of plates 50, 290, and 291 is related to plate 230, as indicated by the dashed and labeled outline that indicates the distal extent of 230. However, these plates each include an additional distal extension of differing length and/or radius to extend farther distally along the medial condyle, producing distinct distal portions 56, 292, or 293. Accordingly, each plate may include an extension of the convex arc described above. Plates 50 and 290, viewed in profile, define an arc having a radius similar to that of plate 230 (see FIGS. 22 and 24). However plate 291 defines an arc with a radius that is about 10% to 30% larger (compare FIGS. 24 and 26). Therefore, sets of medial condyle plates may include distal portions that extend different lengths over the medial condyle, have a greater number of openings in the distal portion, and/or that define arcs of different radius. Different radii may be suitable for medial condyles of distinct size, different amounts of soft tissue separating the plate from bone, and/or the like.

Distal portions 292, 293 may extend distally from the arc that they define. Distal extensions 294, 296, 298 may include regions that are generally orthogonal, in profile, to long axis 260, as shown in FIGS. 22, 24, and 26, respectively. In some embodiments, such as plates 290 and 291, distal extensions 296, 298 may cross long axis 260, thus extending across a length-width plane defined by long axis 260 and transverse axis 298. Alternatively, or in addition, distal extensions 296, 298 may bend counter to the bend of the arc from which they extended more proximally, to produce a distal tab or foot 302, 304. Tabs 302, 304 may be formed by a substantially orthogonal bend that defines a concave arc with respect to the outer surface and the length, so that the tab defines a second length-width plane that is generally parallel to the first length-width plane defined by axes 260, 298, as shown in FIGS. 24 and 26. The first and second length-width planes may be spaced somewhat from each other. Tabs 302, 304 each may include one or more openings to receive a fastener for attachment to the medially facing surface of the trochlea. The one or more openings may be separated by a spacer region 306 from a group of openings disposed more centrally on the distal portion, as shown in FIGS. 23 and 25.

VIII. Posterior Humerus Bone Plates

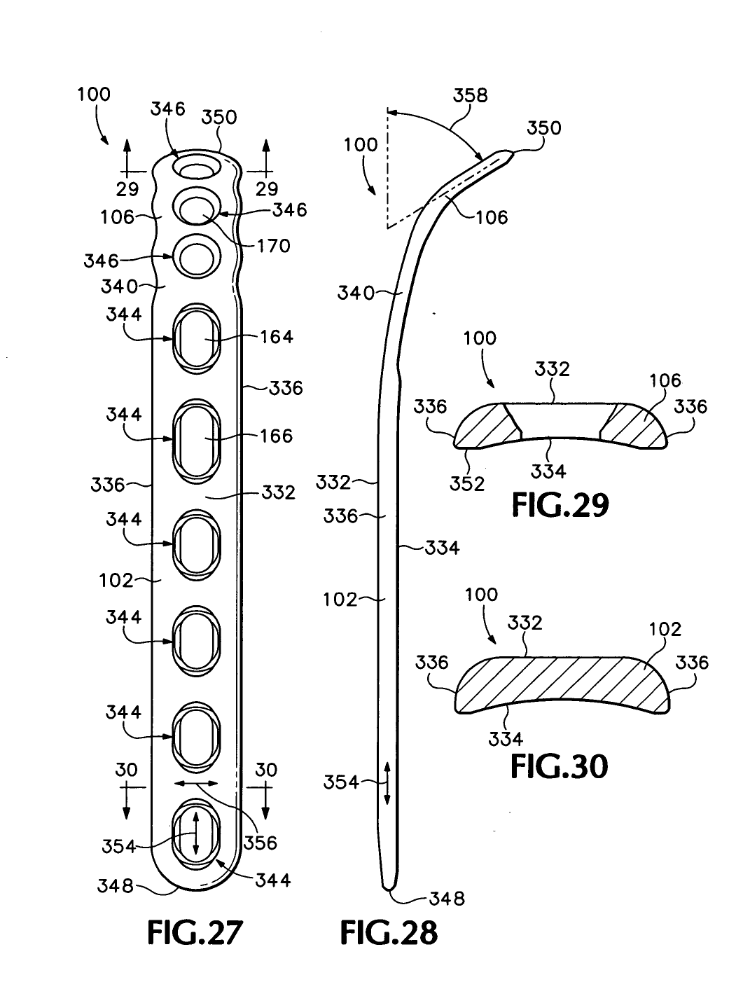

This section describes posterior plates configured for fixing fractures of the posterior-lateral periarticular and/or shaft regions of the left and/or right distal humerus; see FIGS. 27-30.

FIGS. 27-30 show top, side and cross-sectional views, respectively, of posterior plate 100 (see also FIG. 3). Plate 100 includes an outer (or bone-opposing) surface 332, an inner (or bone-facing) surface 334, and sides 336. Proximal (or shaft-anchor) portion 102 extends to join bridge (or intermediate) portion 340, which in turn joins to distal (or end-anchor) portion 106. Proximal and distal portions define sets of openings 344, 346, and extend in generally opposite directions to proximal and distal ends 348, 350.

Posterior plates in general, and plate 100 in particular, may include any suitable features described above for the lateral and/or medial condyle plates (or described below for the olecranon and coronoid plates). For example, as shown by FIGS. 28-30, posterior plate 100 may thin toward distal portion 106. Alternatively, or in addition, plate 100 may vary in width, for example, having narrowed regions intermediate openings of set 346. Accordingly, plate 100 may have a varying cross-section or cross-sectional area. Plate 100 may thin near close to proximal end 348. Plate 100 may include openings positioned and sized as described above, such as elongate openings 164, 166 in proximal portion 102, circular openings 170 in distal portion 106. Chamfers 352 may be included on inner surface 334.

Posterior plates may be handed or lack handedness. Accordingly, posterior plates may be configured for fixing fractures of either the left or right distal humerus (but not both). Alternatively, as shown for posterior plate 100, posterior plates may be configured for use on each of a left and right distal humerus. FIG. 27 shows that posterior plate 100 may be generally linear when viewed from outer surface 332, orthogonal to a length-width plane defined by long axis 354 and transverse (width) axis 356. Posterior plate 100 may be bilaterally symmetrical in outline and/or in opening placement. Here, centers of openings 164, 166, and 170 define a line when the plate projected onto to the length-width plane (see FIG. 27). Other plates may have this arrangement of openings, for example, see the medial condyle plates shown in FIGS. 16, 21, 23, and 25.

FIG. 28 shows how posterior plates may be contoured in profile. Plate 100 may extend linearly from proximal end 348, along long axis 354 and through some or all of proximal portion 102. Plate 100 may bend to define a convex arc with respect to outer surface as the plate transitions to bridge portion 340 and distal portion 106. The convex arc may include two arcs: a proximal convex arc and a distal convex arc. The proximal convex arc directs bridge portion 340 in profile distally at an angle of about 3 degrees to 20 degrees, about 5 degrees to 15 degrees, or about 8 degrees from long axis 354. The distal convex arc directs distal portion distally at an angle 358 relative to long axis 354 of about 35 degrees to 75 degrees, about 45 degrees to 65 degrees, about 50 to 60 degrees, or about 55 degrees. Accordingly, the distal convex arc may have a substantially larger radius than the distal convex arc, or at least about two-fold greater.

IX. Olecranon Bone Plates

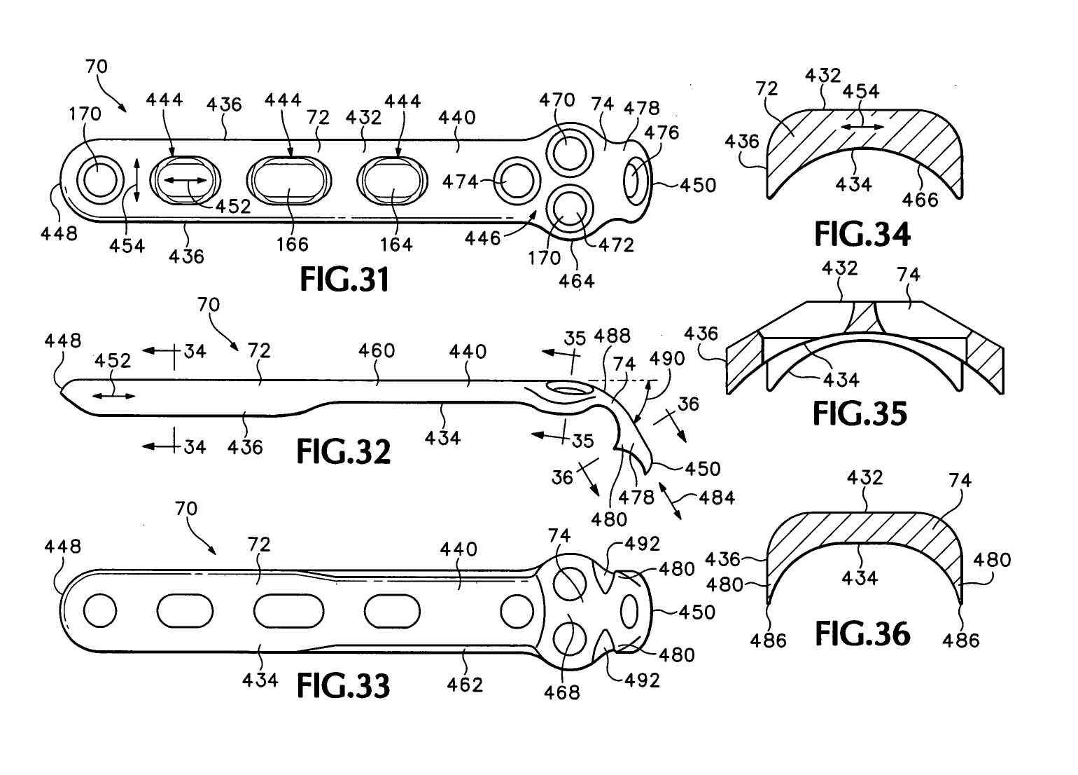

This section describes olecranon plates for fixing fractures of periarticular and/or shaft regions of the left and/or right proximal-posterior ulna, particularly the olecranon; see FIGS. 31-42.

FIGS. 31-36 show top, side, bottom, and two cross-sectional views, respectively, of a Smaller-sized olecranon plate 70 (see also FIGS. 1 and 2). Plate 70 includes an outer (or bone-opposing) surface 432, an inner (or bone-facing) surface 434, and sides 436. Distal (or shaft-anchor) portion 72 extends to join to bridge (or intermediate) portion 440, which in turn joins to proximal (or end-anchor) portion 74. Distal and proximal portions define opening sets 444, 446, respectively, and extend in generally opposite directions to distal and proximal ends 448, 450. Plate 70 also may be described relative to a set of generally orthogonal axes: a long axis 452 defined by distal portion 72, a transverse or width axis 454, and a thickness axis 456. Each axis may be related, generally by rotation, to a corresponding local axis when the plate bends, for example, tangents for local axes corresponding to length and thickness.

Please note that the olecranon and coronoid plates described below extend in a generally “reversed” orientation on bone relative to the plates described above. Thus, for these plates, the end-anchor portion is attached more proximally on bone than the shaft-anchor portion. Accordingly, proximal and distal nomenclatures refer here and throughout to relative positions for intended attachment to bone.

Olecranon plates in general, and olecranon plate 70 in particular, may include any suitable features described above for the other bone plates. For example, olecranon plate 70 may include elongate openings 164, 166 in distal portion 72 and/or may include circular openings 170 in proximal and/or distal portions, such as a single opening 170 near distal end 448. Some or all of the openings in proximal portion 74 may be more closely spaced than openings in distal portion 70, as described above for lateral plate 120. Alternatively, or in addition, plate 70 may thin proximally in the end-anchor portion, at the sides and/or central positions, as shown in FIGS. 34-36, and as also described above for lateral condyle plate 120. For example, sides 436 with decreased thickness, shown at 460, are produced as plate 70 extends along distal portion 72 toward and into bridge region 440 (see FIG. 32). Decreased thickness may be produced by flanking chamfers 462, which may extend generally coplanar to each other (see FIG. 33). Inner surface 434 may be generally tubular, for example, having a substantially constant radius of curvature (measured transversely) throughout distal portion 72 and bridge portion 440. Distal end 448 may be somewhat tapered distally, in thickness and/or width, as described above for other plates.

Plate 70 may include a widened region 464 in proximal portion 74. Widened region 464 generally includes any region that has a greater width than the average width of distal portion 70. Region 464 may be disposed near or at a position along the length at which plate 70 bends away from long axis 452 (see below). Region 464 may have a greater radius of curvature on inner surface 434, measured transversely, than a tubular recess 466 in distal portion 70, as shown by a comparison of FIGS. 34 and 35. Widened region also may be curved or bent somewhat along its length (generally along long axis 452), thus producing a recess 468 on inner surface 434. Recess 468 may be concave generally along long axis 452 and transverse axis 454. Accordingly, recess 468 may fit at or near the ridge formed by the olecranon at its posterior-proximal junction.

Plate 70 may include an opening configuration suited for more effective olecranon fixation. For example, one or more of the openings in opening set 446 may be disposed off-center. Off-center means that the opening is not centered between sides 436, that is, the opening is disposed laterally. Here, plate 70 includes plural off-center openings 470, 472. Off-center openings may be disposed transversely, that is centers of openings 470, 472 may define a line that is generally parallel to transverse axis 454, or may be disposed obliquely, for example, in a staggered configuration. In plate 70, openings 470, 472 may be spaced closely to a centered opening 474 to form a triangular cluster of central openings in an intermediate region of the proximal portion. However, any clustered arrangement may be suitable. Alternatively, or in addition, olecranon plates may include one or more proximal openings, such as opening 476 disposed more closely to proximal end 450. A proximal opening(s) may be defined by an angled or end region 478 of proximal portion 74 (described below), so that the proximal opening(s) defines a plane that is disposed obliquely or orthogonally to planes defined by the openings of the intermediate region, such as off-center openings 470, 472.

Plate 70 may include one or plural spacing members or projections, such as prongs 480, disposed near proximal end 450 on angled region 478. Prongs 480 may project generally orthogonal to a length-width plane defined locally by angled portion 478 and/or generally orthogonal to a plane defined by proximal opening 476. Thus, prongs 480 may project generally orthogonal to both transverse axis 454 and to a local long axis 484 (see FIG. 32). Prongs 480 may be disposed at positions that are aligned with opening 476 of angled region 478 along local long axis 484, or may be disposed proximally or distally relative to this opening. Prongs generally are defined by inner surface 434 and may be produced by thickened regions along side 436, as shown in FIG. 32, and/or by an elliptical or circular cross section along transverse axis 454, shown in FIG. 36. Prongs may be sharp or somewhat rounded at tips 486. Furthermore, prongs 480 may be suitable for gripping the triceps tendon or other soft tissue, among others, either operatively, during plate 70 positioning, and/or after final positioning. Prongs 480 may produce less compression of the triceps tendon by generally spacing angled region 478 from bone. Spacing means that a region of inner surface 434 in angled region 478 is held in spaced relation to the olecranon surface.

Olecranon plates may be nonhanded or handed (see below). Olecranon plates may have an inner-surface contour that allows the plates to substantially match a bone contour of the proximal-posterior ulna. Accordingly, the bone contour may have or may generally lack handedness. Olecranon plates, such as plate 70, may be generally linear when viewed from outer surface 432, generally orthogonal to a length-width plane defined by long axis 452 and transverse axis 454, as shown in FIG. 31. Plate 70 may have a perimeter and/or an opening configuration that is substantially symmetrical bilaterally to produce a substantially nonhanded plate.

FIG. 32 shows how an olecranon plate may bend in profile. Proximal portion 74 of plate 70 may bend away from long axis 452 to define a convex arc or bend 488 with respect to outer surface 432 and generally along the length. Bending of angled portion distally relative to long axis, to define local long axis 484, may be by an angle 490 of about 40 to 80 degrees, 50 to 70 degrees, or about 60 degrees. This allows inner surface 434 to match a bone contour, from posterior to proximal along the olecranon, that subtends an obtuse angle, or an angle of about 120 degrees. Proximal portion 74 may include a narrowed or thinned region that preferentially bends to facilitate pre- or peri-operatively modifying angle 490 of a precontoured plate. Here, proximal portion 74 is narrowed at an opening-free zone positioned along the length between intermediate openings 470, 472, 474 and proximal opening 476. In addition, proximal portion 74 has a locally thinned region, in this case formed by laterally disposed recesses 492 on inner surface 434. Recesses 492 are produced by central to lateral tapering transversely. However, proximal portion 74 may be thinned at a local region in any suitable manner to produce a preferred site of bending, for example, a groove or channel that extends transversely, among others.

FIGS. 37-42 show top and side views of modified olecranon plates 520, 530, 540. Each Of plates 520, 530, 540 includes a section related to olecranon plate 70 but has a proximal or distal portion that is modified relative to proximal and/or distal portions of plate 70.

Plate 520 has a modified distal portion 552 and proximal portion 554 relative to corresponding portions of plate 70; see FIGS. 37 and 38. Distal portion 552 is extended relative to distal portion 72 and includes an additional opening. In addition, distal portion 552 may thin less along its length as the portion extends proximally, particularly along side 436, shown at 556, relative to distal portion 72, shown at 460 of FIG. 32. Proximal portion 554 includes an angled or end region 558 that may extend at an angle similar to angled region 478 of plate 70 (see above). However, angled region 558 may be longer than angled region 478 shown in FIG. 32, being configured to extend farther proximally and anteriorly along the olecranon when applied to bone. Due to its increased length, angled region 558 may include an increased number of openings, for example, in a transversely centered, linear array as shown. Angled region 558 also may include regions that direct bending positioned at plural sites along local long axis 560, for example, produced by locally narrowed regions 562 or thinned regions (not shown), such as recesses 492 of FIG. 33. The regions that direct bending may be disposed intermediate openings within proximal portion 554, as described above.

Plate 520 and olecranon plates in general may be formed with or without spacing members (projections) or prongs, such as prongs 480 of plate 70. Here, plate 520 lacks prongs. Prongs may not be necessary or suitable for plate 520 because this plate may be used more frequently with severely comminuted olecranon fractures. In such fractures, soft tissue, such as the triceps tendon, may be removed more completely during surgery so that this tissue is no longer disposed between the plate and bone. As a result, plate 520 also may require less thinning along the sides in distal portion 552, as shown at 556.

Olecranon plate 530 has a modified distal portion 572, but has a proximal portion substantially equivalent to proximal portion 74 of plate 70; see FIGS. 39 and 40. Distal portion 572 includes an extended linear portion, allowing plate 540 to fix a greater length of diaphyseal bone on the ulna and may include an increased number of openings, such as elongate openings 164 (or 166) or circular openings (not shown).

Olecranon plate 540 is related to plate 530 but includes an extended distal portion 574; see FIGS. 41 and 42. Distal portion 574 may include additional openings, as shown, to maintain a relatively constant density of openings in distal portion 574. Distal portion 574 may be linear in profile (viewed side-on), as shown in FIG. 42. However, distal portion 574 may be nonlinear when viewed from outer surface 576, generally orthogonal to a length-width plane defined by long axis 578 and transverse (width) axis 580, as in FIG. 41. Accordingly, in contrast to the shorter plates described above, plate 540 has a handedness, being configured for use on a left ulna. Distal portion may define at least two distinct long axes 578, 582 that are bent by about 2 to 10 degrees or about 5 degrees relative to each other, parallel to an axis 584 along which thickness is defined. This rotation defines a nonlinear distal portion 574 that bends rightward as the plate extends from distal to proximal, as viewed from the outer surface for a left-handed embodiment (as in FIG. 41). Such a nonlinear configuration may allow distal portion 574 to track one side of the proximal-posterior ulna as the ulna bends distally.

X. Coronoid Bone Plates

This section describes embodiments of coronoid plates configured for fixing fractures of periarticular and/or shaft regions of the left and/or right proximal-anterior ulna, particularly the coronoid; see FIGS. 43-48. Coronoid bone plates may include any suitable features described above for other bone plates.

FIGS. 43-45 show top, side, and bottom views, respectively, of a left-handed embodiment of a smaller-sized coronoid plate 80. A right-handed embodiment may be configured as a substantial mirror-image replicate (not shown). Plate 80 includes an outer (or bone-opposing) surface 632, an inner (or bone-facing) surface 634, and generally concave and convex sides 636, 637, respectively bistal (or shaft-anchor) portion 82 extends to join bridge portion 640, which in turn joins to buttress portion 84. Distal portion 82 defines opening set 644, which generally includes plural openings 32, such as circular openings 170 and/or elongate openings 164, 166. Distal and buttress portions 82, 84 extend outwardly to distal and proximal ends 648, 650, respectively. Plate 80 also may be described relative to a set of generally orthogonal axes in a central region 651 of distal portion 82: a long (or length) axis 652, a transverse or width axis 654, and a thickness axis 656 (orthogonal to the page on FIG. 43). Each axis may be related, generally by rotation, to a corresponding local axis produced through bending of the plate.

Buttress portion 84 stabilizes and supports the coronoid. Portion 84 may be configured to lie generally parallel to a distally-facing, anterior surface of the coronoid, with generally convex side 637 disposed more anteriorly and/or proximally than generally concave side 636. Buttress portion 84 may be configured to use contact between buttress portion 84 and the coronoid as a primary method of fixation by this portion. Accordingly, buttress portion 84 may be substantially or completely free of openings or may use openings in an auxiliary fixation role. The buttress portion may resist separation from the coronoid process at least partially through the attachment of distal portion 82 to the shaft region with fasteners. To support bone, buttress portion may include one or more spacing members or projections, such as prongs 90. Plate 80 includes two prongs, however three or more projections may be suitable in some cases. Prongs 90 may be included in inner surface 634 and may project toward bone and generally orthogonal to a length-width plane defined by the buttress portion. Here, prongs 90 are formed as thickened regions of generally convex side 637. However, projections may extend from any suitable side or internal region of the inner surface of buttress portion 84. Prongs may include sharp or rounded tips.

Coronoid plate 80 may be configured to be attached to any suitable side of the ulna, for example, the medial side, in order to support the distally facing side of the coronoid. Thus, plate 80 may be precontoured so that its inner surface substantially conforms to distinctly oriented sides of the ulna by twisting and/or bending. Twisting and/or bending of plate 80, either during its construction and/or application, may be enabled by decreased rigidity regions 660, 662. Decreased rigidity region 660 may correspond generally to bridge portion 640, and region 662 may separate openings and/or region 662 may separate central region 651 from distal end 648 of shaft-anchor portion 82. Each region may be formed, for example, by locally narrowing and/or thinning plate 80, as described above for other plates.

FIGS. 46 and 47 show end views of plate 80, illustrating how buttress portion 84 and distal region 664, respectively, may be twisted relative to central region 651. Portion 84 and region 664 may be twisted with the same helical handedness relative to central region 651. Accordingly, proximal and distal ends 648, 650 each may be twisted along an axis or axes that are generally parallel to long axis 652 or obliquely oriented relative to both long axis 652 and transverse axis 654. In this left-handed embodiment, twisting is in a clockwise direction as each end extends toward central region 651, as shown in FIGS. 46 and 47. Twisting may be substantially similarly as the plate extends from distal to proximal ends 648, 650, accordingly each end may be related to central region 651 by a substantially similar angle 666 of approximately 20 to 60 degrees, 30-50 degrees, or about 40 degrees. Accordingly, portion 84 and region 664 may define planes that are rotated relative to each other by the sum of these rotations, approximately 80 degrees, to dispose these planes generally orthogonal to each other. Although plate 80 is configured for a left ulna, helical twist in plate 80 is right-handed, thus rotating clockwise as the plate extends between distal and proximal ends. A mirror-image plate for the right ulna may include a left-handed helical twist. In alternative embodiments, angle 666 may differ between ends so that one end is bent or twisted more relative to central region 651 than the other end.

Plate 80 may be shaped like a twisted crescent, having a generally concave side 636 and a generally convex side 637. Such concave and convex assignments exclude local variations in width produced, for example, by narrowed regions 660, 662. When viewed along axis 656 from outer surface 632, as in FIG. 43, left-handed plate 80 may include a generally linear distal portion 82 that arcs to the right as the plate extends from distal to proximal, through buttress portion 84. Buttress portion 84 may define a local long axis 668 near distal end 650 that is generally orthogonal to long axis 652. As shown in FIG. 2, distal portion 82 may be configured to extend obliquely relative to the long axis of the ulna shaft, for example, at an angle of about 20 to 60 degrees.

FIG. 48 shows a bottom view of an alternative embodiment of a coronoid plate for the left ulna, plate 680. Coronoid plate 680 is a larger version of plate 80 that may be suitable, for example, to fix more extensive injury of the ulna shaft region. Plate 680 may be structured as an extension of plate 80, as indicated by the distal extent of plate 80 shown dashed and labeled. Plate 680 has an extended distal portion 682, which may be structured as an initially arcing, but then generally linear extension of distal portion 82 of plate 80. Distal portion 682 may include elongate openings, circular openings, or a combination thereof. For example, elongate openings may be segregated to distal-end portion 684, and circular openings may be segregated to central portion 686. Buttress portion 84 may be equivalent in plates 80 and 680. Distal portion 682 may be configured to extend obliquely relative to the long axis of the ulna in central portion 686, but then bend or arch to parallel the long axis of the ulna distally in distal-end portion 684.

XI. Exemplary Uses of Periarticular Elbow Plates

These plates and associated fasteners may be selected in some instances according to how well they satisfy one or more of the following technical objectives: (1) ensuring that as many screws as possible pass through a plate, (2) ensuring that as many screws as possible engage a fragment on the opposite side that also is fixed to a plate, (3) ensuring that as many screws as possible are placed in distal fragments, (4) ensuring that each screw engages as many articular fragments as possible, (5) ensuring that each screw is as long as possible, (6) ensuring that plates are applied such that compression is achieved at the supracondylar level for both columns, and (7) ensuring that plates are strong enough and stiff enough to resist breaking or bending before union occurs at the supracondylar level.

The bone plates may be used as follows. The discontinuity in the bone (e.g., the humerus and/or the ulna) may be reduced by appropriate means, including manually. A suitable bone plate may be selected and positioned through a surgical incision so that a portion of the plate spans the discontinuity and a portion contacts the reduced bone on opposite sides of the discontinuity. The bone plate may be formed to mate with the bone before and/or during fixation. Holes may be drilled in the bone, and the bone plate may be secured to the bone using suitable fasteners such as bone screws passing through openings in the plate and the holes in the bone. After the bone is sufficiently healed, the bone plate and fasteners may be removed, or they may be left in place to avoid (temporary) reductions in strength of the bone. Patients with broken bones may be anesthetized during reduction, fixation, and removal to minimize discomfort.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in applications claiming priority from this or a related application. Such claims, whether directed to a different invention or to the same invention, and whether broader, narrower, equal, or different in scope to the original claims, also are regarded as included within the subject matter of the inventions of the present disclosure.

We claim:

1. A set for bone fixation, comprising:
at least two bone plates, each bone plate including a distinct color that indicates a distinct bone region to which the bone plate is configured to be secured.

2. The set of claim 1, wherein the distinct color indicates a left half or a right half of a skeleton in which the distinct bone region is disposed.

3. The set of claim 2, further comprising at least one other bone plate configured for use on each of the left half and the right half of a skeleton, the at least one other bone plate including a color that is distinct from the distinct colors of the at least two bone plates.

4. The set of claim 2, wherein the distinct color also indicates a particular bone region within one of the left half and the right half of the skeleton.

5. The set of claim 1, wherein each of the distinct bone regions is included in the same bone.

6. The set of claim 5, wherein the distinct bone regions are overlapping.

7. The set of claim 5, wherein the distinct bone regions are nonoverlapping.

8. The set of claim 1, wherein each distinct bone region is included in a different bone.

9. The set of claim 1, wherein each distinct bone region is included in one of a distal region of a humerus bone and a proximal region of an ulna bone.

10. The set of claim 1, wherein at least one of the bone plates includes at least one prong configured to engage bone when the bone plate is secured to bone.

11. The set of claim 1, wherein at least one of the bone plates has a first portion configured to be secured adjacent an end of a long bone and a second portion configured to be secured toward or on a shaft of the long bone, and wherein the first portion of the bone plate is thinner on average than the second portion of the bone plate.

12. The set of claim 1, wherein at least one of the bone plates includes a curved bone-facing surface configured to match the curved contour of the distinct bone region to which the at least bone plate is configured to be secured.

13. The set of claim 1, further comprising instructions that relate each bone plate to the distinct bone region to which the bone plate is configured to be secured.

14. A method of bone fixation, comprising:
selecting a distinct bone region for fixation;
selecting one of the at least two bone plates of claim 1 according to the distinct color of the one bone plate that indicates the distinct bone region; and
securing the one bone plate to the distinct bone region.

15. A bone plate for bone fixation, comprising:
a first portion defining a plurality of openings configured to receive bone screws for securing the first portion to a bone; and
a second portion joined to the first portion and including at least one prong configured to engage the bone when the first portion is secured to the bone.

16. The bone plate of claim 15, wherein the at least one prong is two or more prongs.

17. The bone plate of claim 15, the second portion defining a plane, wherein the at least one prong extends at least substantially orthogonal to the plane.

18. The bone plate of claim 15, wherein the prong includes a pointed tip configured to penetrate the bone when the first portion is secured to the bone.

19. The bone plate of claim 15, wherein the second portion includes a bone-facing surface, and wherein the at least one prong includes a blunt tip configured to engage a surface of the bone so at least a region of the bone-facing surface is spaced from the bone when the first portion is secured to the bone.

20. The bone plate of claim 15, wherein the second portion defines one or more openings configured to receive bone fasteners for securing the second portion to the bone.

21. The bone plate of claim 15, wherein the first and second portions are configured to be disposed generally on opposing sides of a bone discontinuity, and wherein the second portion has no openings for receiving bone fasteners.

22. The bone plate of claim 15, wherein the bone plate is configured for fixation of a proximal region of an ulna bone.

23. The bone plate of claim 15, wherein the bone plate is configured for fixation of at least one of an olecranon and a coronoid process of an ulna bone.

24. The bone plate of claim 15, wherein one of the first and second portions is thinner on average than the other of the first and second portions, and wherein the thinner of the first and second portions is configured to be secured to an end of a long bone and the thicker of the first and second portions is configured to be secured toward or on the shaft of the long bone.

25. The bone plate of claim 15, wherein at least one of the first and second portions includes a curved bone-facing surface configured to match the curved contour of a surface of the bone.

26. A method of bone fixation, comprising:
selecting a bone plate according to claim 15; and
securing the bone plate to the bone.

27. The method of claim 26, further comprising a step of placing the bone plate on the bone, prior to the step of securing, such that the at least one prong positions the second portion of the bone plate away from the bone, and such that at least one of a tendon, a nerve, and a blood vessel lies between the second portion and the bone.

28. The method of claim 26, wherein the step of securing the bone plate to the bone includes a step of penetrating the bone with at least one of the at least one prongs.

29. A bone plate for fixing a discontinuity in a long bone, the long bone having a shaft and two ends, the bone plate comprising:
a first portion configured to be secured adjacent an end of the long bone; and
a second portion configured to be secured toward or on the shaft of the long bone;
wherein the first portion is thinner on average normal to the surface of the bone as secured than the second portion.

30. A method of bone fixation, comprising:
selecting a bone plate according to claim 29; and
securing the bone plate to the bone.
